(19) United States
(12) Patent Application Publication
(10) Pub. No.: US 2009/0069561 A1
(43) Pub. Date: Mar. 12, 2009

(54) CLICK CHEMISTRY ROUTE TO TRIAZOLE DENDRIMERS

(75) Inventors: Valery Fokin, Oceanside, CA (US); K. Barry Sharpless, La Jolla, CA (US); Peng Wu, Berkeley, CA (US); Alina Feldman, New York, NY (US)

Correspondence Address: Olson & Cepuritis, LTD., 20 NORTH WACKER DRIVE, 36TH FLOOR, CHICAGO, IL, 60606, US

(73) Assignee: The Scripps Research Institute, La Jolla, CA (US)

(21) Appl. No.: 11/631,081

(22) PCT Filed: Jun. 30, 2005
(86) PCT No.: PCT/US2005/023718

§ 371 Date: Nov. 12, 2008

Related U.S. Application Data
(60) Provisional application No. 60/584,817, filed on Jun. 30, 2004.

(51) Int. Cl. C07D 249/04; C07C 247/04; C07D 403/12; C07C 43/205; C07C 317/10; C07C 205/03; C07D 251/32; C07C 247/06

(52) U.S. Cl. 544/192; 552/10; 552/11; 548/255; 544/366; 568/649; 568/307; 568/30; 977/754

(57) ABSTRACT

The high efficiency and fidelity of click chemistry permits a large number of diverse dendrimers encompassing a wide variety of functionalities at the chain ends, repeat units, and/or core to be prepared. Almost quantitative yields were obtained during the synthesis. In some cases, filtration or solvent extraction was the only method required for purification. These features represent a significant advancement in dendrimer chemistry and demonstrate an evolving synergy between organic chemistry and functional materials.

### TECHNICAL FIELD

The invention relates to dendrimers and to methods for making dendrimers. More particularly, the invention relates to the use of click chemistry for synthesizing triazole dendrimers.

### BACKGROUND

Unique properties of dendrimers, which are a direct consequence of their regular structure, have sparked significant interest in recent years (D. A. Tomalia, et al. Angew. Chem. 1990, 102, 119-57; Angew. Chem., Int. Ed. 1990, 29, 138; D. A. Tomalia, H. D. Durst in Topics in Current Chemistry, Vol. 165 (Eds.: E. Weber), Springer-Verlag, Berlin, 1993; pp. 193-313; F. Zeng, S. C. Zimmerman Chem. Rev. 1997, 97, 1681-1712; C. J. Hawker in Advances in Polymer Science, Vol. 147, Springer-Verlag, Berlin, Heidelberg, Germany, 1999, pp. 113-160; M. Fischer, F. Vogtle Angew. Chem. 1999, 111, 934-955; Angew. Chem., Int. Ed. 1999, 38, 884-905; A. W. Bosman, et al. Chem. Rev. 1999, 99, 1665-1688; J.-P. Majoral, A.-M. Caminade Chem. Rev. 1999, 99, 845-880; L. J. Twyman, et al. Chem. Soc. Rev. 2002, 31, 69-82; J. M. J. Fréchet Proc. Natl. Acad. Sci. USA, 2002, 99, 4782-4787). A large number of dendritic structures varied in size, solubility, and function have been prepared. However, most dendrimer syntheses, particularly at later generations, require high monomer loading, tedious and lengthy chromatographic separations, and generate considerable waste (S. M. Grayson, J. M. J. Frechet Chem. Rev. 2001, 101, 3919-3967; J. M. J. Frechet J. Polym. Sci., Polym. Chem. 2003, 41, 3713-3725). For example, synthesis of polyether dendrimers (S. C. Zimmerman, et al. J. Am. Chem. Soc. 2003, 125, 13504-13518; S. Kimata, et al. J. Polym. Sci., Polym. Chem. 2003, 41, 3524-3530; A. Dahan, M. Portnoy Macromolecules 2003, 36, 1034-1038; E. M. Harth, et al. J. Am. Chem. Soc. 2002, 124, 3926-3938; F. S. Precup-Blaga, et al. J. Am. Chem. Soc. 2003, 125, 12953-12960), based on a sequence of Williamson etherifications and halogenations, suffers from incompatibility with various functional groups and complicated purifications. Protic functional groups at the periphery, such as —OH, —COOH and —NH₂, are not compatible with Williamson etherifications and halogenations.

Dendrimers have many uses. For example, the recently discovered ability of polydentate 1,4-disubstituted 1,2,3-triazoles ligands to stabilize Cu(I) species even in aqueous aerobic conditions (T. R. Chan, et al. Org. Lett. Submitted), has already proven crucial in biological applications (Q. Wang, et al. J. Am. Chem. Soc. 2003, 125, 3192-3193; A. E. Speers, et al. J. Am. Chem. Soc. 2003, 125, 4686-4687; A. J. Link, D. A. Tirrell J. Am. Chem. Soc. 2003, 125, 11164-11165; A. Deiters, et al. J. Am. Chem. Soc. 2003, 125, 11782-11783). Triazole dendrimers can be employed for this purpose. Also, dendrimers are employable for making porous materials, i.e., the dendrimers are mixed with a matrix material, the matrix material is solidified, and the dendrimers are vaporized. In order to achieve a precise porosity, dendrimers of uniform size must be employed. Unfortunately, prior to the present disclosure, it was not practical to make dendrimers having a precisely uniform size, i.e., substantially all dendrimers having the same size.

What is needed is a simple method for making dendrimers of precisely uniform size.

### SUMMARY

A highly efficient route for the production of triazole-based dendrimers employs click chemistry. This route benefits from the unprecedented reliability of the Cu(I)-catalyzed ligation of terminal acetylenes and azides. The chemistry is highly regioselective, resulting in 1,4-disubstituted triazoles. A variety of functional groups are compatible with the process and the only major byproduct formed in the reaction is NaCl. All second generation and some third generation dendrons were directly isolated as pure solids (i.e. no chromatographic separations), meeting the requirements for large scale applications.

One aspect of the invention is directed to a process for producing a product dendron having a single azide group. The process comprises a first step wherein “n” organic azide molecules are reacted with an ABₙ molecule. The ABₙ molecule has “n” terminal acetylene functionalities and one halomethyl group, where “n” is two or greater. The reaction occurs in the presence of sufficient copper catalyst to insure complete reaction for producing a product molecule having “n” triazoles and one halomethyl group. In the second step of the process, the product molecule of the first step is reacted with sufficient sodium azide in an organic/aqueous solvent mixture at a temperature high enough to give complete or nearly complete displacement of the chloride from the halomethyl group for producing the product dendron having a single azide group. In a preferred mode, the product dendron is a first generation dendron. Preferred organic azides are selected from the group represented by the following structures:

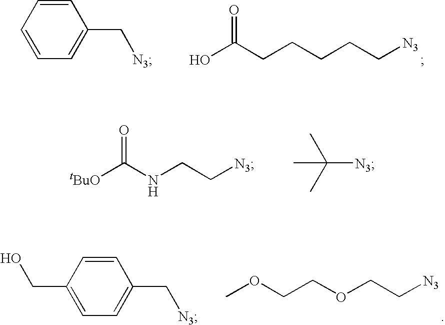

In another preferred mode, “n” is two. Preferred ABₙ molecules are selected from the group represented by the following structures:

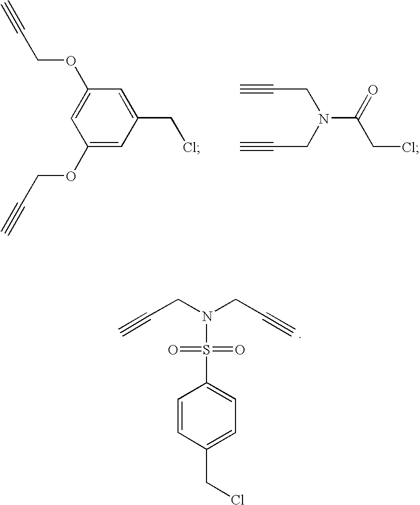

In another preferred mode, the product dendron is a second generation dendron and each of the “n” organic azide molecules is a first generation dendron. In this instance, the first generation dendron may also be made by the process of the invention or not. In another preferred mode, the product dendron is a third generation dendron and each of the “n” organic azide molecules is a second generation dendron. In this instance, the second generation dendron may also be made by the process of the invention or not. In another preferred mode, the product dendron is a fourth generation dendron and each of the “n” organic azide molecules is a third generation dendron. In this instance, the third generation dendron may also be made by the process of the invention or not.

Another aspect of the invention is directed to a process for producing a triazole containing dendrimer. The process comprises the step of reacting two or more dendrons, each dendron possessing a single azide functionality, with a polyacetylene core compound, the polyacetylene core compound containing two or more terminal acetylene groups, in a suitable solvent and in the presence of catalytic quantity of copper(I) species for catalyzing a triazole formation reaction for forming the dendrimer. Optionally, the process may include the further step of washing the product of the first step with sufficient aqueous ammonium hydroxide/citrate solution to remove copper species that may be bound to triazole moieties of the dendrimer. In a preferred mode of this aspect of the invention, the polyacetylene core is selected from the group represented by the following structures:

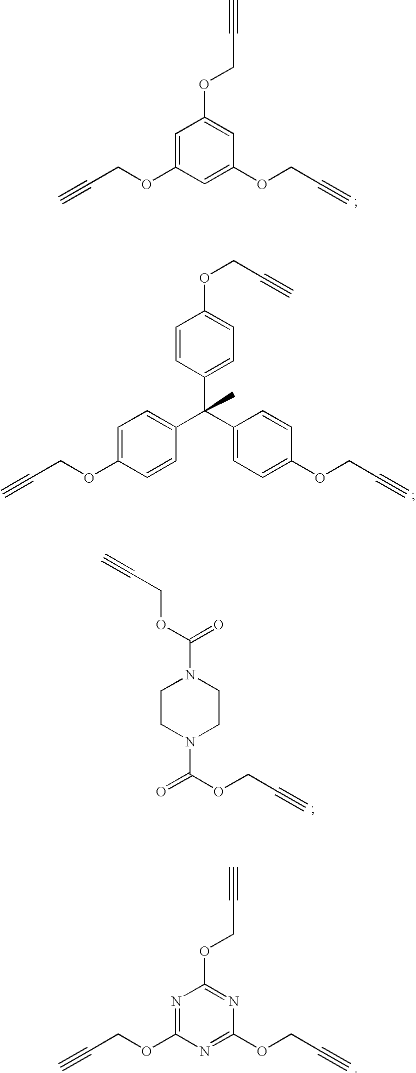

This process may be employed for making a first, second, third, or fourth generation dendrimer wherein the dendron is first, second, third, or fourth generation dendron, respectively.

Another aspect of the invention is directed to the first, second, third, and fourth dendrimers made according to the above processes.

Another aspect of the invention is directed to a trifunctional reagent represented by the following formula:

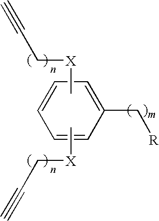

In the above structure, X is a diradical selected from the group consisting of —O— and —S—; R is a radical selected from the group consisting of —Cl and —Br; n is 1-10; and m is 1-10. A preferred embodiment of this aspect of the invention may be represented by the following formula:

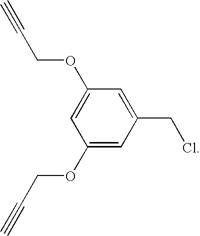

Another aspect of the invention is directed to a trifunctional reagent represented by the following formula:

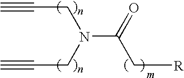

In the above structure, R is a radical selected from the group consisting of —Cl and —Br; n is 1-1.0; and m is 1-10. A preferred embodiment of this aspect of the invention may be represented by the following formula:

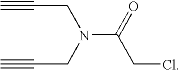

Another aspect of the invention is directed to a trifunctional reagent represented by the following formula:

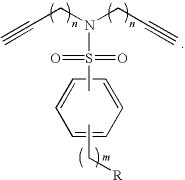

In the above structure, R is a radical selected from the group consisting of —Cl and —Br; n is 1-10; and m is 1-10. A preferred embodiment of this aspect of the invention may be represented by the following formula:

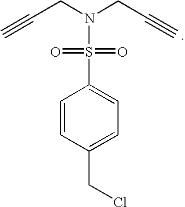

Another aspect of the invention is directed to a core molecule represented by the following formula:

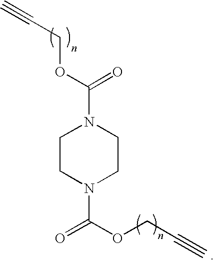

In the above structure, n is 1-10.

### BRIEF DESCRIPTION OF FIGURES

FIG. 1 illustrates an example of a large dendrimer that can be prepared by the method outlined.

FIG. 2 illustrates the copper(I)-catalyzed synthesis of 1,4-disubstituted 1,2,3-triazoles.

FIG. 3 illustrates the reaction sequence by which the individual branches or dendrons, were constructed, starting from the “outside” of the molecule.

FIG. 4 illustrates three structures that were chosen for the AB₂ monomers.

FIG. 5 illustrates the different monoazides which are used for the chain ends.

FIG. 6 illustrates an NMR spectrum of the product bis-triazole.

FIG. 7 illustrates the GPC traces for the crude reaction products, MEE-B-[G-4]-N₃ (9d), MEE-B-[G-3]-N₃ (6d), and MEE-B-[G-2]-N₃ (4d), obtained by dendritic growth from the benzyl ether monomer 11, and the azido di(ethylene glycol) derivative 19.

FIG. 8 illustrates the structures of the polyacetylene cores to which the dendrons were anchored.

FIG. 9 illustrates a representative example of a dendrimer that is obtained by coupling a third generation dendron to a triacetylene core.

FIG. 10 illustrates a MALDI-TOF mass spectrum of dendrimer 7a.

### DETAILED DESCRIPTION

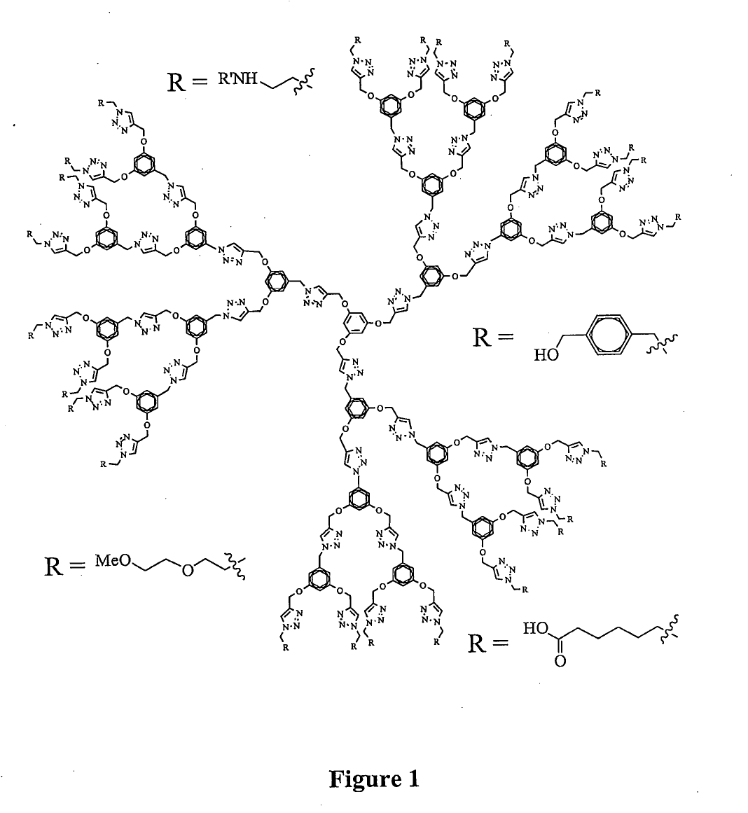

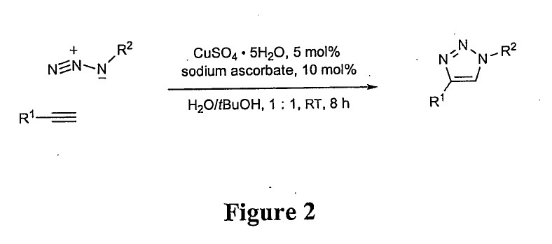

A highly efficient route for the production of triazole-based dendrimers employs click chemistry. This new efficient and preparatively simple route results in the generation of diverse dendritic structures (FIG. 1) of high purity and in excellent yield. The unique aspects of this route arise from the near perfect reliability of the Cu^{I} catalyzed synthesis of 1,2,3-triazoles from azides and alkynes (FIG. 2) (V. V. Rostovtsev, et al. Angew. Chem. Int. Ed. 2002, 41, 2596-2599; C. W. Tornøe, et al. J. Org. Chem. 2002, 67, 3057). The reaction is experimentally simple—proceeding well in aqueous solutions without protection from oxygen, requiring only stoichiometric amounts of starting materials, and generating virtually no byproducts. Equally important is the wide scope of this transformation, high selectivity and nearly quantitative yields. Not surprising for the best click reaction to date (H. C. Kolb, et al. Angew. Chem. Int. Ed. 2001, 40, 2004-2021), the procedure involves simply mixing and stirring, whereupon pure products are isolated by filtration or simple extraction.

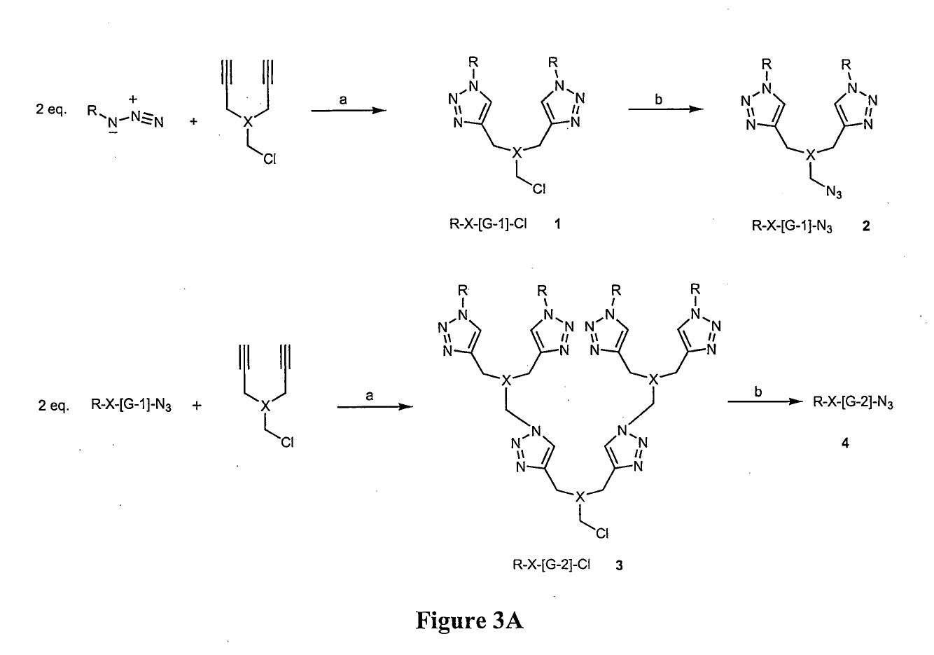

Frechét's convergent approach was utilized in the dendrimer synthesis disclosed herein (C. J. Hawker, J. M. J. Fréchet J. Am. Chem. Soc. 1990, 112, 7638-7647). Thus, the individual branches, or dendrons, were built sequentially, starting on the “outside” of the molecule. They were then coupled to a multivalent centerpiece (“core”) in the last step, as outlined in FIGS. 3A and 3B, which led to a variety of dendrimers with different chain end groups (R) and internal repeat units (X).

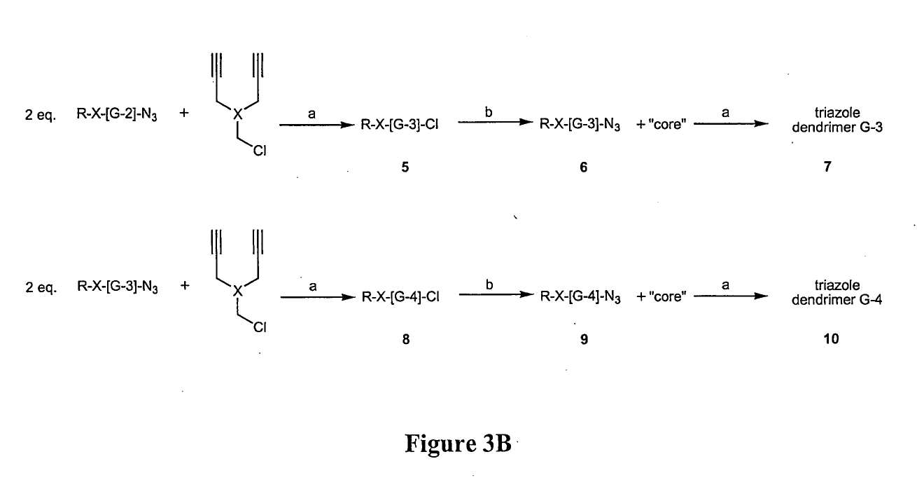

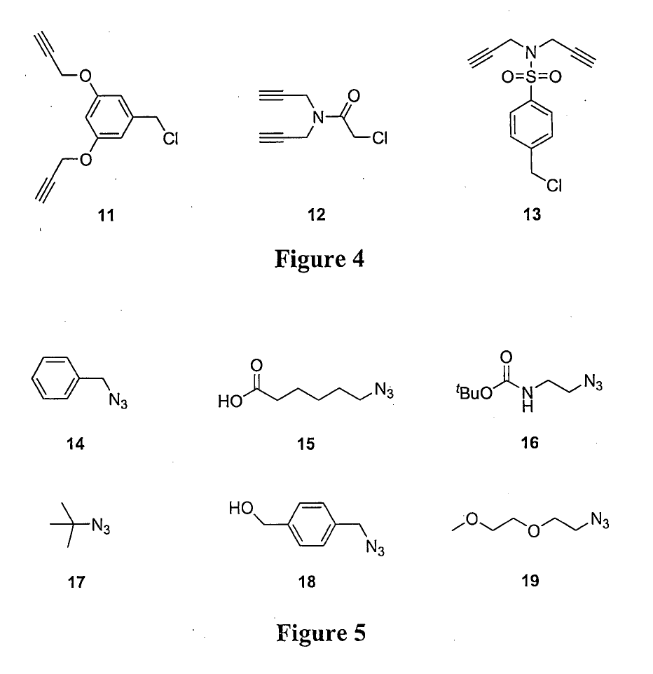

In exploiting Cu(I) catalyzed reaction for dendrimer construction, a variety of AB₂ monomers can be envisaged based on terminal acetylenes and alkyl halide functionalities. Readily available starting materials and simple synthetic strategies for the introduction of acetylenic groups lead to significant structural diversity (FIG. 4). However, one structural feature was retained between 11, 12, and 13, namely the presence of a single chloromethyl group. This was specifically incorporated into the synthetic strategy disclosed herein to allow facile activation of the focal point group during dendrimer construction by the convergent growth approach. Reaction of dendritic fragments containing a single chloromethyl group with sodium azide leads to quantitative formation of the desired azidomethyl group, which can then be coupled with 11, 12, or 13 to give the next generation dendron (FIGS. 3A and 3B). As with AB₂ monomer units, a variety of the chain ends with reactive functional groups ranging from carboxylic acid to alcohol can be employed in the construction of diverse triazole dendrimers (FIG. 5).

Copper(I)-catalyzed reactions of AB₂ monomers and chain end units were carried out in the presence of 2-5 mol % CUSO₄, 5-10 mol % sodium ascorbate in 1:1 mixture of water and tert-butyl alcohol at room temperature, generating the desired bis-triazoles in near quantitative yield. The trace amount of copper salts in the products was easily removed by washing with an ammonium hydroxide-citrate aqueous buffer. The high degree of efficiency permitted a stoichiometric amount (2.0 equivalents) of azides to be used. When combined with the absence of side products, purifications were greatly simplified. This is in direct contrast with a classical polyether dendrimer synthesis by the convergent growth approach where an excess of dendron (2.05-2.20 equivalents) is typically used to increase yields of the next generation dendritic fragment. Furthermore, purification by flash chromatography is usually required at each step (C. J. Hawker, J. M. J. Fréchet J. Am. Chem. Soc. 1990, 112, 7638-7647).

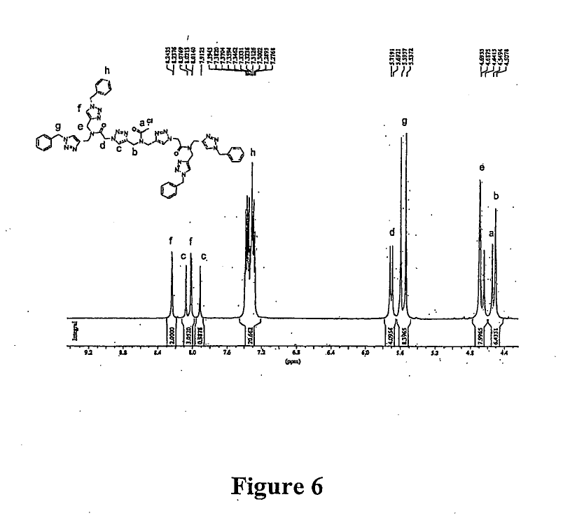

In the next step, the primary chlorides were converted to the corresponding azides by reaction with 1.5 equivalents of sodium azide in acetone/water mixture, which was equally facile and typically resulted in yields of more than 95% with the only byproduct being NaCl. (Monochloride dendrons generated from benzenesulfonamide 13 were converted into the corresponding azides in wet DMF.) The dendrons were then ‘grown’ via the reaction of the resulting azides with the original monomers 11, 12 or 13. All second generation dendrons were isolated as pure white solids by simple filtration or aqueous workup, producing the second generation azide dendrons in isolated yields exceeding 90%. As seen in the crude ¹H NMR spectrum of dendron Bn-F-[G-2]-Cl (3b) (FIG. 6), the integration ratio for proton f and c is 2:1 which, in comparison with the mixture obtained from a purely thermal process, indicates a 100% regioselectivity.

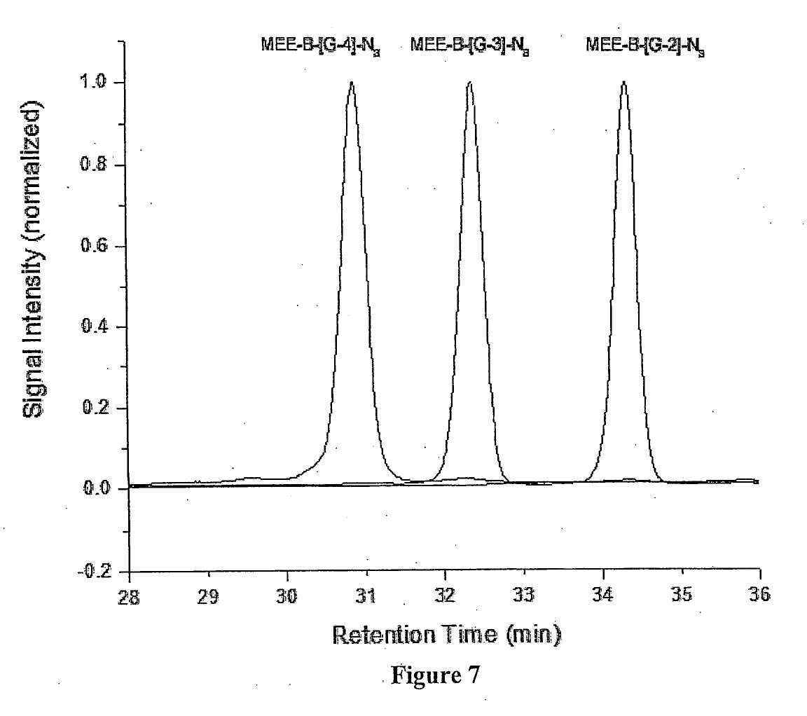

Following the same procedure, amide monomer 12 with tert-butyl azide 17 at the periphery as well as the benzyl ether monomer 11 with azide 19 were propagated to the fourth generation. Monomer 12 with the azide 16 and 19 at the periphery was propagated to the third generation, respectively. In those cases where dendrimers were not soluble in aqueous mixtures, slight modification of reaction conditions led to the same degree of efficiency and near quantitative yields. For example, benzyl terminated dendrimers prepared from 14 and 11 were found to be insoluble in 1:1 H₂O/THF solutions at second generation, resulting in no reaction. Similarly, conversion of the chloromethyl group to the azide group was unsuccessful with aqueous sodium azide. To overcome this difficulty, the copper-catalyzed reaction was performed in THF with an organosoluble Cu(I) species, (PPh₃)₃CuBr, under microwave irradiation, leading to quantitative yields of the next generation dendritic fragment. Similar results for azide formation were obtained when the displacement was performed with sodium azide in DMF or DMSO. Analysis of the dendrons by MALDI-TOF mass spectroscopy as well as GPC provides no signs of products with defects that would arise from incomplete branches (FIG. 7).

Works by Tomalia, Fréchet, and others demonstrate solubility properties of dendritic molecules are dominated by their periphery (S. M. Grayson, J. M. J. Frechet Chem. Rev. 2001, 101, 3919-3967; J. M. J. Fréchet J. Polym. Sci., Polym. Chem. 2003, 41, 3713-3725). Similarly, observations of the dendrimers disclosed herein largely follow these trends. A unique property of these triazole-based dendrons is, as the molecules reach higher generations, they become less soluble in ethyl acetate and slightly more soluble in dichloromethane, chloroform, alcohols and, surprisingly, aqueous mixtures. (In general, dendrons generated from benzenesulfonamide 13 are less soluble than dendrons made from acetamide 12 in all solvents tested, while the incorporation of 19 as a chain end increases the solubility substantially.)

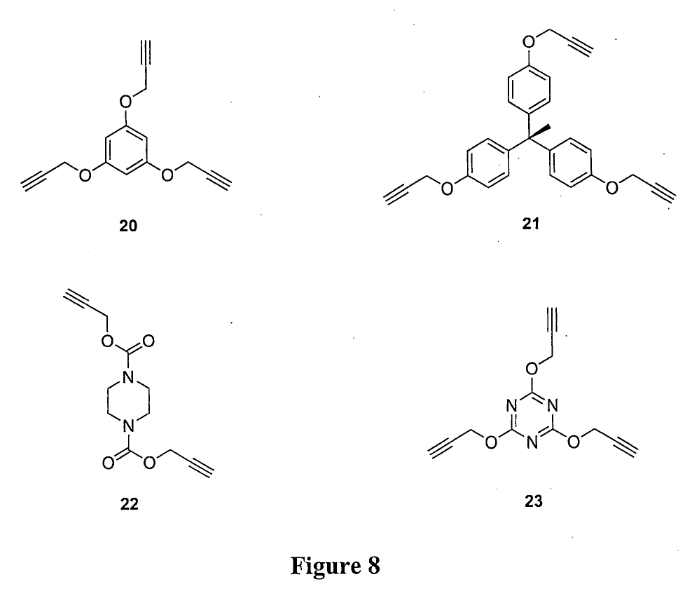

Finally, several third and fourth generation triazole dendrimers were constructed by anchoring these dendrons to a variety of polyacetylene cores (FIG. 8).

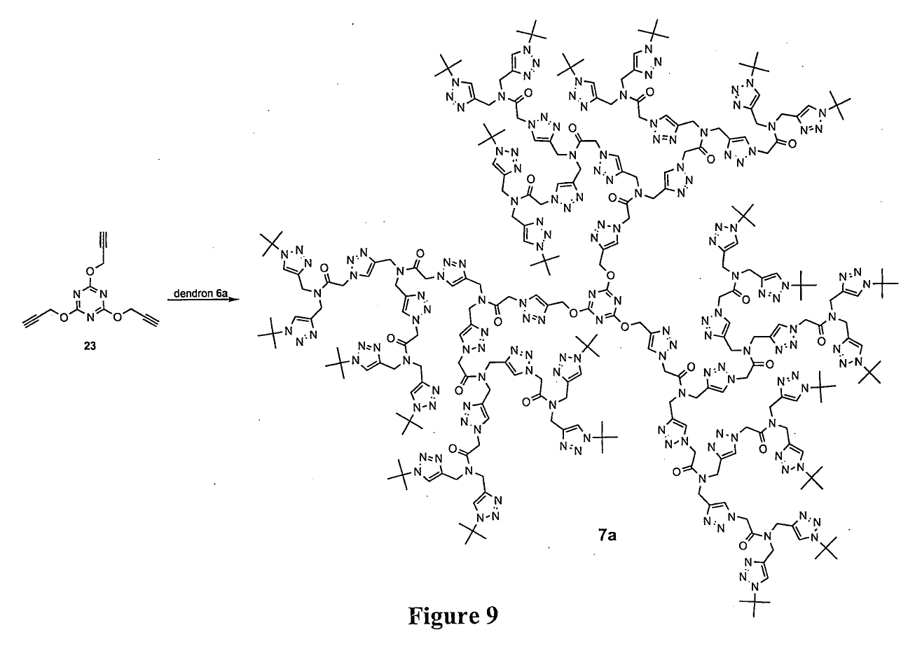

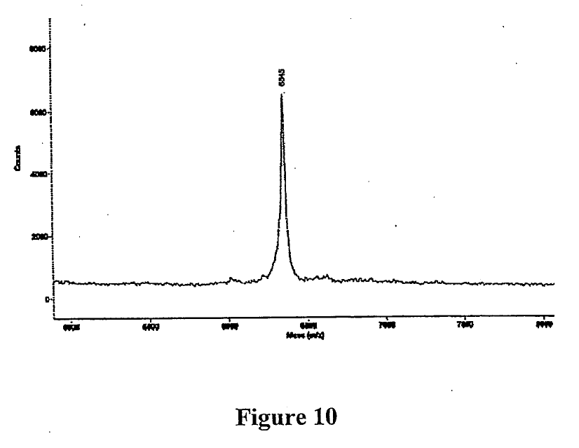

A representative example is shown in FIG. 9, where the third generation dendron tBu-F-[G-3]-N₃ (6a) was coupled directly with 2,4,6-tris-prop-2-ynyloxy-[1,3,5]triazine (23) in the presence of the in situ generated Cu(I) catalyst. It is noteworthy that even at these low concentrations (0.06 M in alkyne and azide), the catalysis proceeded rapidly enough at room temperature, reaching completion in less than 30 hrs as indicated by LC-MS analysis. Dendrimer 7a, with 24 periphery units (molecular weight 6322 Da), was isolated as a white solid in 92% yield. All dendrimers were characterized by ¹H and ¹³C NMR, and further confirmation of structure and purity was obtained by GPC and MALDI-TOF mass spectroscopy (FIG. 10). High polarity, good alcohol/water mixture solubility, and strong UV absorptions at 210 and 229 nm are unique features of this new class of triazole dendrimers.

### DETAILED DESCRIPTION OF FIGURES

FIG. 1 shows an example of a large dendrimer that can be prepared by the method outlined. The different R groups shown allow for different solubilities of the resultant dendrimer.

FIG. 2 shows the copper(I)-catalyzed synthesis of 1,4-disubstituted 1,2,3-triazoles. The copper(I) is obtained by in situ reduction of the copper(I) species, here obtained from copper sulfate. The reaction is run at ambient temperature in a water/alcohol solvent mixture to give nearly quantitative yields of the 1,2,3-triazole product.

FIGS. 3A and 3B show the reaction sequence by which the individual branches or dendrons, were constructed, starting from the “outside” of the molecule. These are then coupled to a multivalent centerpiece or “core” in the last step. The internal repeat units are “X” and the chain end groups are “R.”

FIG. 4 shows three structures that were chosen for the AB₂ monomers. These were based on terminal acetylenes and alkyl halide functionalities. The structural feature, in addition to the diacetylene, that was retained between the three structures 11, 12, and 13 was the chloromethyl group. The reaction of dendritic fragments containing one chloromethyl group with sodium azide would lead to the quantitative formation of the azidomethyl group which would then be coupled with 11, 12 or 13 to give the next generation dendron.

FIG. 5 shows the different monoazides which are used for the chain ends. There are reactive and non-reactive end groups for the dendrimers. The non-reactive groups have an aryl, alkyl and methoxyethoxy ends and the reactive end groups have carboxylic acid, benzyl alcohol and protected primary amine functionalities.

FIG. 6 is an NMR spectrum of the product bis-triazole. The spectrum shows the complete lack of any regioisomers in the product as would be expected from a thermal cycloaddition reaction. The ratio of integration for protons f and c is 2:1 which shows that only one regioisomer is formed in the cycloaddition. The presence of two signals for both f and c protons is due to the different magnetic environments in the amide bond rotomers.

FIG. 7 shows the GPC traces for the crude reaction products, MEE-B-[G-4]-N₃ (9d), MEE-B-[G-3]-N₃ (6d), and MEE-B-[G-2]-N₃ (4d), obtained by dendritic growth from the benzyl ether monomer 11, and the azido di(ethylene glycol) derivative 19. These traces show no signs of products with defects stemming from incomplete branches.

FIG. 8 shows the structures of the polyacetylene cores to which the dendrons were anchored.

FIG. 9 is a representative example of a dendrimer that is obtained by coupling a third generation dendron to a triacetylene core.

FIG. 10 is a MALDI-TOF mass spectrum of dendrimer 7a. This time-of-flight mass spectrum was part of the proof of purity of this product.

### EXPERIMENTAL SECTION

General Methods. Commercial reagents were obtained from Aldrich and were used without further purification. Deuterated solvents were purchased from Cambridge Isotope Laboratories, Inc. Analytical TLC was performed on commercial Merck Plates coated with silica gel GF254 (0.24 mm thick). Silica Gel for flash chromatography was Merck Kieselgel 60 (230-400 mesh, ASTM). NMR (¹H, ¹³C) spectra were recorded either on a Bruker AMX-400, AMX-500 or AMX-600 MHz spectrometer. Coupling constants (J) are reported in hertz, and chemical shifts are reported in parts per million (δ) relative to CHCl₃ (7.26 ppm for ¹H and 77.2 ppm for ¹³C) or DMSO (2.50 ppm for ¹H and 39.5 ppm for ¹³C) or CD₃OD (3.31 ppm for ¹H and 49.0 ppm for ¹³C) or acetone (2.05 ppm for ¹H. and 29.9 ppm for ¹³C) as internal reference. Preparation HPLC was performed on a Dynamax HPLC system using a ZORBAX SB-C18 column (21.2 mm i.d.×25 cm) with H₂O/CH₃CN as eluent, the flowing rate was 6.5 ml/min. Gel Permeation Chromatography was performed in tetrahydrofuran (THF) on a Waters chromatograph equipped with four 5-μm Waters columns (300 mm×7.7 mm) connected in series with increasing pore size (two mixed B, 10³ Å, 10⁵ Å). A Waters 410 differential refractometer and a 996 photodiode array detector were employed. The molecular weights of the polymers were calculated relative to linear polystyrene standards. The modulated differential scanning calorimetry (MDSC) measurements were performed with the TA Instruments DSC 2920 and a ramp rate of 4 degrees per minute. The thermal gravimetric analysis measurements were done with the TA Instruments Hi-Res TGA 2950, under nitrogen purge, and the ramp rate was 10 degrees per minute. 2-chloro-N,N-di(prop-2-ynyl)acetamide 12 (A. J. Speziale, P. C. Hamm, J. Am. Chem. Soc. 1956, 78, 2556-2229); azide 15 (D. Charon, M. Mondange, J.-F. Pons, K. Le Blay, R. Chaby, Bioorg. Med. Chem., 1998, 6, 755-765), 16 (P. G. Mattingly, Synthesis 1990, 366-368), 17 (J. C. Bottaro, P. E. Penwell, R. J. Schmitt, Syn. Comm. 1997, 27, 1465-1467); 1,3,5-tris(prop-2-ynyloxy)benzene 20 (P. Place, R. Pepin, in FRXXBL FR 2598408 A1 19871113 FR. 1987), 1,1,1-tris(4-(prop-2-ynyloxy) phenyl)ethane 21 (D. O'Krongly, S. R. Denmeade, M. Y. Chiang, R. Breslow, J. Am. Chem. Soc. 1985, 107, 5544-5545) were prepared according to the reported methods.

The nomenclature used for the dendritic framework is as follows: R-X-[G-n]-Y, where R describes the functional groups at periphery, Bn for benzyl, Boc for tert-butyl ethylcarbamate, tBu for tert-butyl, MEE for (2-methoxyethoxy)ethane; X describes internal repeat units, B for 1, 3 dioxybenzene, F for formamide, S for benzenesulfonamide; n is the number for generations; Y describes functional group at the focal point, either chloride, Cl, or azide, N₃.

### Syntheses of Repeating Units.

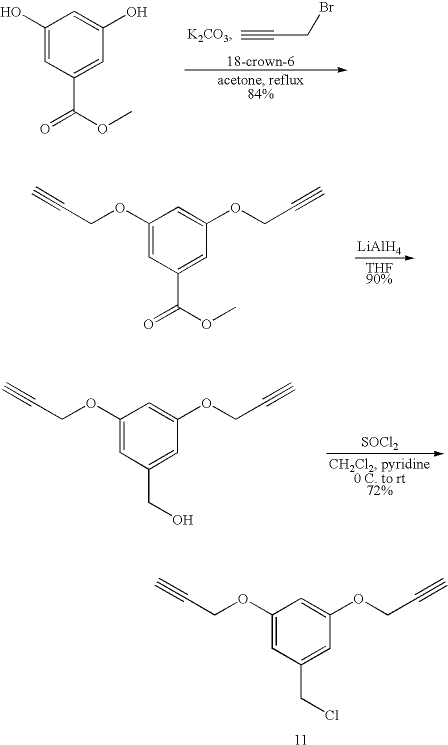

Methyl 3,5-Bis(propargyloxy)benzoate. To a stirred solution of methyl 3,5-dihydroxybenzoate (16.8 g, 100 mmol) and propargyl bromide (29.7 g, 220 mmol) in acetone (300 ml) were added potassium carbonate (15.1 g, 109 mmol) and 18-crown-6 (0.1 g, 0.4 mmol). The reaction mixture was heated at reflux under nitrogen for 24 hours, filtered and evaporated to dryness. The crude material was then crystallized in methanol to give the ester as pale yellow crystals (20.6 g, 84.4%). ¹H NMR (500 MHz, CDCl₃): δ=2.55 (t, J=2.4 Hz, C≡CH, 2H), 3.92 (s, CH₃O, 3H), 4.73 (d, J=2.4 Hz, CH₂C≡CH, 4H), 6.83 (s, p-Ar, 1H), 7.31 (s, o-Ar, 2H). ¹³C NMR (125 MHz, CDCl₃): δ=52.76 (s, CH₃O, 1C), 56.51 (s, CH₂C≡CH, 1C), 76.38 (s, C≡CH, 1C), 78.34 (s, C≡CH, 1C), 107.91 (s, p-Ar, 1C), 109.27 (s, o-Ar, 2C), 132.54 (s, CCOOCH₃, 1C), 158.90 (s, m-Ar, 2C), 166.86 (s, COOCH₃, 1C).

3,5-Bis(propargyloxy)benzyl alcohol. To a stirred solution of the ester (20.6 g, 84.4 mmol) in anhydrous THF (170 ml) was added lithium aluminum hydride (3.99 g, 105 mmol) in small portions. Beckstrom's reagent (25 g) was then added to quench the remaining lithium aluminum hydride. The reaction mixture was filtered under vacuum, the solid was rinsed with dichloromethane and the filtrate dried with MgSO₄. After evaporation of the solvents, the alcohol was recovered as white crystals (16.4 g, 90.1%). ¹H NMR (500 MHz, CDCl₃): δ=2.46 (t, J=2.4 Hz, C≡CH, 2H), 4.45 (s, CH₂OH, 2H), 4.61 (d, J=2.4 Hz, CH₂C≡CH, 4H), 6.46 (s, p-Ar, 1H), 6.56 (s, o-Ar, 2H). ¹³C NMR (500 MHz, CDCl₃): δ=56.30 (s, CH₂C≡CH, 1C), 65.50 (s, CH₂OH, 1C), 76.09 (s, C≡CH, 2C), 78.76 (s, C≡CH, 2C), 101.88 (s, p-Ar, 1C), 106.60 (s, o-Ar, 2C), 143.97 (s, CCH₂OH, 1C), 159.23 (s, m-Ar, 2C).

3,5-Bis(propargyloxy)benzyl chloride, 11. To a stirred solution of the alcohol (14.7 g, 68.0 mmol) in dichloromethane (200 ml) and pyridine (10.7 g, 136.0 mmol) was added and the mixture was placed on an ice bath. Thionyl chloride (12.1 g, 102 mmol) dissolved in dichloromethane (20 ml) was added dropwise to the reaction mixture and the ice bath was allowed to warm to room temperature. The reaction mixture was then allowed to stir under Ar for 24 h followed by quenching with water. The organic layer was allowed to separate and was washed with water (3×100 ml), dried over MgSO₄, filtered, and evaporated to dryness. The crude product was purified by flash chromatography, loading with 1:1 dichloromethane:hexane and eluting with 2:1 dichloromethane:hexane to give the chloromethyl monomer, 1, as a white solid; ¹H NMR (500 MHz, CDCl₃) δ=2.57 (t, 2H, ≡CH), 4.55 (s, 2H, CH₂Cl), 4.73 (d, 4H, CH₂O), 6.59 (t, 2H, ArH), and 6.80 (d, 1H, ArH).

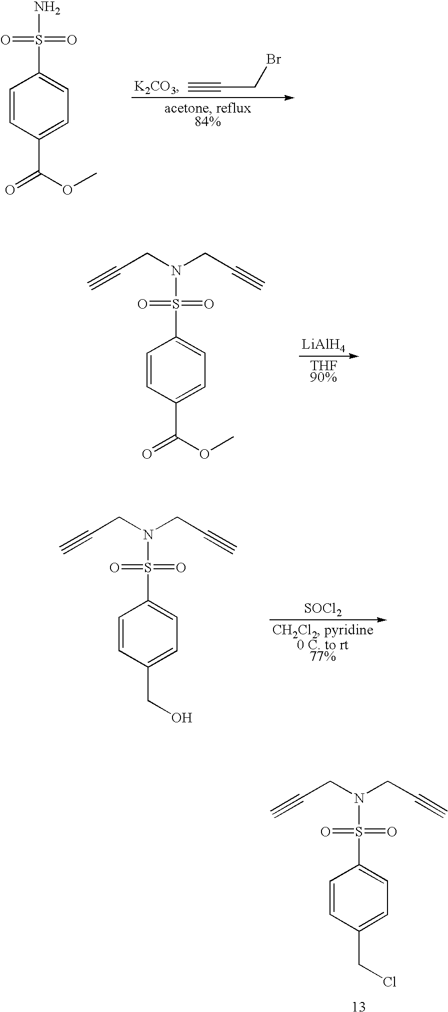

4-(Chloromethyl)-N,N-di(prop-2-ynyl)benzenesulfonamide, 13. The compound was prepared using a similar procedure as for 11. ¹H NMR (400 MHz, CDCl₃): δ 2.15 (t, J=2.4 Hz, C≡CH, 2H), 4.17 (d, J=2.4 Hz, CH₂C≡CH, 4H), 4.61 (s, CH₂Cl, 2H), 7.52 (d, J=6.4 Hz, Ar—H, 2H), 7.82 (d, J=6.4 Hz, Ar—H, 2H). ¹³C NMR (100 MHz, CDCl₃): δ=36.4 (s, CH₂C≡CH, 2C), 45.1 (s, CH₂Cl, 1C), 74.4 (s, C≡CH, 2C), 76.1 (s, C≡CH, 2C), 129.2 (s, Ar—C, 2C), 129.3 (s, Ar—C, 2C), 138.2 (s, CCH₂Cl, 1C), 142.9 (s, CSO₂, C).

### Syntheses of Monofunctional Azides:

1-Azido-2-(2-methoxyethoxy)ethane, 19. A solution of 1-bromo-2-(2-methoxyethoxy) ethane (12.4 g, 67.8 mmol) and sodium azide (13.2 g, 203 mmol) in water (150 ml) was stirred under reflux for 16 hours. The aqueous phase was extracted with dichloromethane (2×200 ml), dried with MgSO₄ and evaporated to dryness, to give 19 as a colorless oil in 87.3% yield. ¹H NMR (500 MHz, CDCl₃): δ=3.29 (s, CH₃O, 3H), 3.30 (t, J=5.2 Hz, CH₂N₃, 2H), 3.44-3.48 (m, CH₃OCH₂, 2H), 3.53-3.60 (m, CH₂OCH₂, 4H). ¹³C NMR (125 MHz, CDCl₃): δ=50.89 (s, CH₂N₃, 1C), 59.27 (s, CH₃O, 1C), 70.29 (s, CH₃OCH₂CH₂, 1C), 70.84 (s, CH₃OCH₂CH₂, 1C), 72.21 (s, CH₂CH₂N₃, 1C).

### Syntheses of Cores:

Diprop-2-ynyl piperazine-1,4-dicarboxylate, 22. To a 4 ml CH₂Cl₂ solution of propargyl chloroformate (237 mg, 2 mmol) was added piperazine 86 mg at 0° C., followed by dropwise addition of Et₃N. The reaction was then stirred at room temperature for 3 hrs. until LC-MS indicated the completion of the reaction. 5 ml 10% HCl was added and the separated organic phase was then washed with NaHCO₃ (sat.), brine, and dried with Na₂SO₄. After evaporating the solvent, the crude product was purified by flash chromatography (hexane:ethyl acetate 3:1) and gave 22 as a white solid, yield 220 mg (88%). ¹H NMR (600 MHz, [D₆]Acetone): δ=3.03 (t, J=2.6 Hz, C≡CH, 2H), 3.48 (br, NC₂H₄N, 8H), 4.72 (d, J=2.6 Hz, CH₂C≡CH, 4H). ¹³C NMR (150 MHz, [D₆]Acetone): δ=44.3 (s, NC₂H₄N, 4C), 53.4 (s, CH₂C≡CH, 2C), 76.2 (s, CH₂C≡CH, 2C), 79.6 (s, C≡CH, 2C), 154.9 (s, CO, 2C). m.p. 101-102° C.

2,4,6-Tris(prop-2-ynyloxy)-1,3,5-triazine, 23. Propargyl alcohol (10 ml) was added slowly to a suspension of cyanuric chloride (2.2 g, 12.1 mmol) in 15 ml THF at room temperature followed by K₂CO₃ (5.2 g, 36.3 mmol). Reaction heated to 60° C. overnight. The reaction mixture was filtered. After evaporation of solvent, the residue was dissolved in 80 ml CH₂Cl₂, and washed with dilute citric acid (10%), saturated brine. Dried over MgSO₄, evaporated to give 23 as white solid in 90% yield. ¹H NMR (600 MHz, [D₆]Acetone): δ=3.13 (t, J=2.2 Hz, C≡CH, 3H), 5.10 (d, J=2.2 Hz, CH₂C≡CH, 6H). ¹³C NMR (150 MHz, [D₆]Acetone): δ=53.4 (s, CH₂C≡CH, 3C), 77.3 (s, CH₂C≡CH, 3C), 78.4 (s, C≡CH, 3C), 173.5 (s, Ar—C, 3C). m.p. 69-70° C.

A Representative Procedure A for the Cu(I) Catalyzed Triazole Ligation Reaction: 2-Chloro-N,N-di(prop-2-ynyl)acetamide 12 (300 mg, 1.765 mmol) was mixed with 470 mg (3.529 mmol, 2.00 eq.) of benzyl azide 14. The mixture was mixed with 2 ml of 1:1 tBuOH/H₂O solution. Sodium ascorbate (35 mg, 0.177 mmol, 0.10 eq.) was added as a solid, followed by the addition of CuSO₄ (22 mg, 0.089 mmol, 0.05 eq.). The reaction was stirred overnight at room temperature. The white cloudy suspension was diluted with 10 ml H₂O and 1 ml concentrated NH₄OH, stirred for 10 minutes, and then filtered. The resulting filtrate, a white powder, was washed 3 times with 10 ml H₂O and dried to obtain the pure Bn-F-[G-1]-Cl 1b. (737 mg, 96% yield).

A Representative Procedure B for the Cu(I)-Catalyzed Catalyzed Triazole Ligation Reaction: 300 mg (1.765 mmol) of 12 was mixed with 656 mg (3.529 mmol, 2.00 eq.) of Boc-protected azidoethylamine 16. The mixture was mixed with 2 ml of 1:1 tBuOH/H₂O solution. Sodium ascorbate (35 mg, 0.177 mmol, 0.10 eq.) was added as a solid, followed by the addition of CuSO₄ (22 mg, 0.089 mmol, 0.05 eq.). The reaction was stirred overnight at room temperature. The light yellow mixture was diluted with 10 ml H₂O and 1 ml concentrated NH₄OH, stirred for 10 minutes and extracted 3 times with 30 ml portions of EtOAc. The organic layer was washed 2 times with saturated NaCl, dried over MgSO₄, and evaporated to yield pure product Boc-F-[G-1]-Cl, 1c (898 mg, 94% yield).

A Representative Procedure for the Conversion of Dendritic Chlorides to Azides: 500 mg (1.36 mmol) of 1a was dissolved in 4 ml acetone/water (4:1). NaN₃ (132 mg, 2.04 mmol, 1.5 eq.) was added, and the mixture was heated to 60° C. for 1 hour. The mixture is cooled to room temperature, acetone evaporated, diluted with 10 ml H₂O, and extracted 3 times with EtOAc. The organic layer was washed with saturated NaCl, dried over MgSO₄, and evaporated. tBu-F-[G-1]-N₃ 2a was obtained as a white solid (490 mg, 96%).

General Procedure for Non-aqueous Click Chemistry Catalyzed by Cu(PPh₃)₃Br. A solution of 3,5-bis(propargyloxy)benzyl chloride, 11, (234 mg, 1.00 mmol), benzyl azide, 4, (266 mg, 2.00 mmol), N,N-diisopropylethylamine (48 mg, 0.37 mmol) and Cu(PPh₃)₃Br (55 mg, 0.12 mmol) in tetrahydrofuran (5 ml) was submitted to microwave irradiation at 140° C. (nominal temperature) for 5 minutes. The crude product was purified by filtering through a silica plug eluting with a 9:1 mixture of dichloromethane and methanol, to give Bn-B-[G-1]—Cl (1d) as a colorless oil (477 mg, 95.5%). ¹H NMR (500 MHz, CDCl₃): (=4.62 (s, CH₂Cl, 2H), 5.06 (s, CH₂O, 4H), 5.41 (s, CH₂N, 4H), 6.67 (s, ArH, 3H), 7.21-7.37 (m, ArH, 10H), and 8.23 (s, ArH, 2H).

General Procedure for the Non-Aqueous Synthesis of Dendritic Azides. A mixture of the dendritic chloride 1d (500 mg, 1.00 mmol) and sodium azide (325 mg, 5.0 mmol) was dissolved in DMSO (5 ml). The reaction was heated at 60° C. for 24 h, poured into water (200 ml). The aqueous layer was extracted with CH₂Cl₂ (3×50 ml), combined, washed with water (2×50 ml) dried over MgSO₄, and evaporated to dryness. Purification by filtration through a silica plug, eluting with 10% MeOH:EtOAc, gave the pure azidomethyl derivative 2d. Yield: 492 mg, 97.1%. ¹H NMR (500 MHz, CDCl₃): δ=4.36 (s, CH₂Cl, 2H), 5.03 (s, CH₂O, 4H), 5.44 (s, CH₂N, 4H), 6.63 (d, ArH, 2H), 6.68 (t, ArH, 1H), 7.22-7.35 (m, ArH, 10H), and 8.21 (s, ArH, 2H).

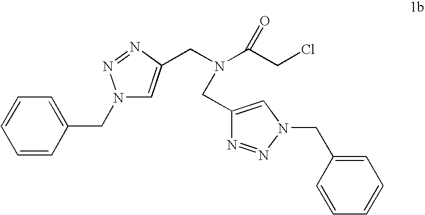

Bn-F-[G-1]—Cl, 1b: ¹H NMR (500 MHz, CDCl₃): δ=7.53 (s, 2H), 7.36 (m, 6H), 7.23 (m, 4H), 5.47 (s, 2H), 5.43 (s, 2H), 4.65 (s, 2H), 4.56 (s, 2H), 4.42 (s, 2H). ¹³C NMR (125 MHz, CDCl₃,): δ=167.1, 134.4, 129.4, 129.1, 54.5, 43.1, 41.9. m.p. 111-112° C.

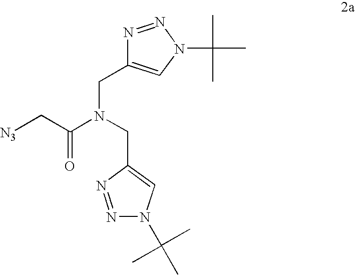

tBu-F-[G-1]-N₃, 2a: ¹H NMR (500 MHz, CDCl₃): δ=7.72 (s, 1H), 7.70 (s, 1H), 4.66 (s, 2H), 4.59 (s, 2H), 4.35 (s, 2H), 1.66 (s, 9H), 1.65 (s, 9H); ¹³C NMR (125 MHz, CDCl₃): δ=163.8, 142.5, 121.9, 120.9, 60.4, 51.6, 43.1, 41.5, 30.6 ppm. Elemental anal Calcd for C₁₆H₂₆N₁₀O (%): C, 51.32, H, 7.00, N, 37.41. Found: C, 51.21, H, 6.95, N, 36.50. m.p. 113-115° C.

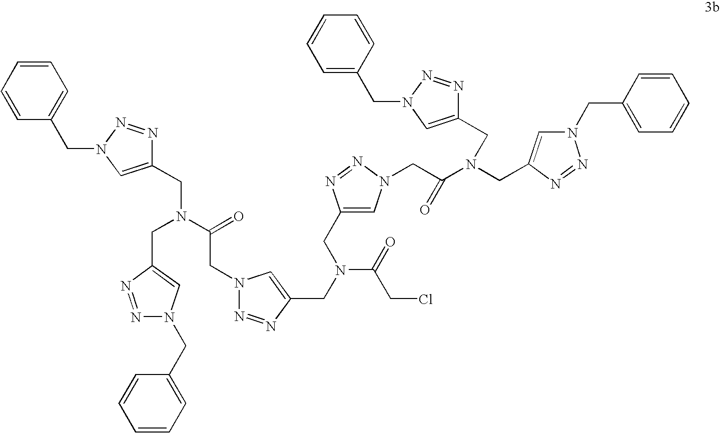

n-F-[G-2]-Cl, 3b: ¹H NMR (500 MHz, [D6]DMSO): δ=8.24 (s, 1H), 8.08 (s, 1H), 8.02 (s, 1H), 7.91 (s, 1H), 7.35 (m, 20H), 5.70 (d, 4H), 5.60 (s, 4H), 5.54 (s, 4H), 4.67 (m, 8H), 4.55 (s, 2H), 4.51 (s, 4H) ¹³C NMR (125 MHz, [D6]DMSO): δ=167.1, 144.1, 137.3, 137.2, 130.1, 129.5, 125.3, 125.2, 54.2, 54.1, 52.3, 44.0, 43.1, 42.6, 32.6. MALDI-TOF: 1076 (MNa⁺), PDI: 1.01.

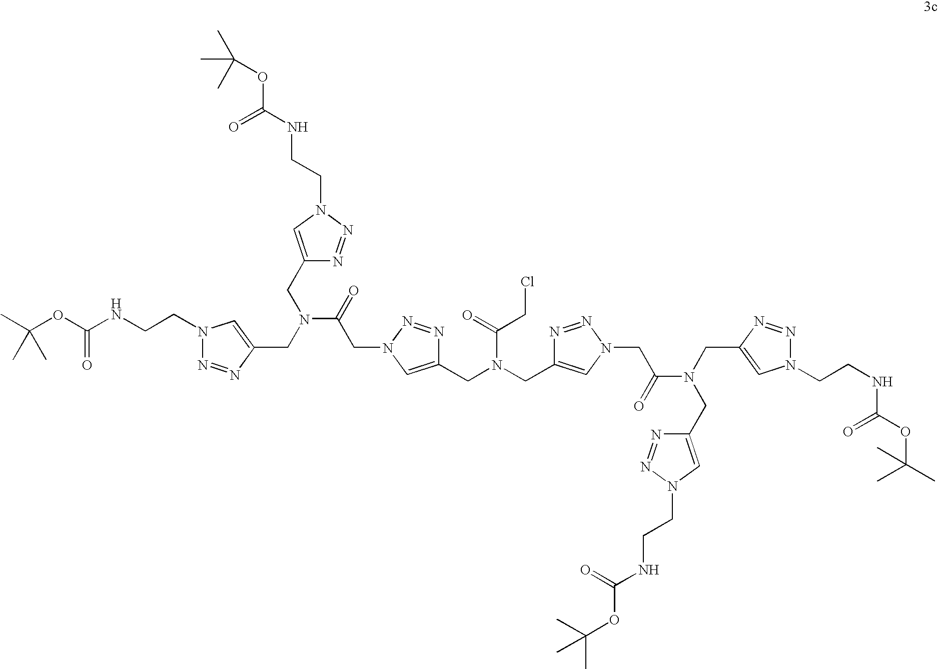

Boc-F-[G-2]-Cl, 3c: ¹H NMR (500 MHz, [D6]Acetone): δ=8.04 (s, 1H), 8.02 (s, 1H), 7.82 (s, 1H), 7.80 (s, 1H), 7.77 (s, 2H), 6.25 (br, 4H), 5.75 (s, 4H), 4.75 (m, 8H), 4.62 (m, 6H), 4.48 (m, 8H), 3.55 (m, 8H), 1.37 (s, 36H). ¹³C NMR (125 MHz, [D6]Acetone): δ 167.1, 166.9, 166.7, 156.8, 144.2, 143.9, 143.7, 126.2, 124.8, 124.7, 124.5, 79.3, 52.1, 50.7, 50.5, 43.5, 43.3, 42.5, 41.5, 28.7 ppm. MALDI-TOF: 1267 (MH⁺), 1289 (MNa⁺).

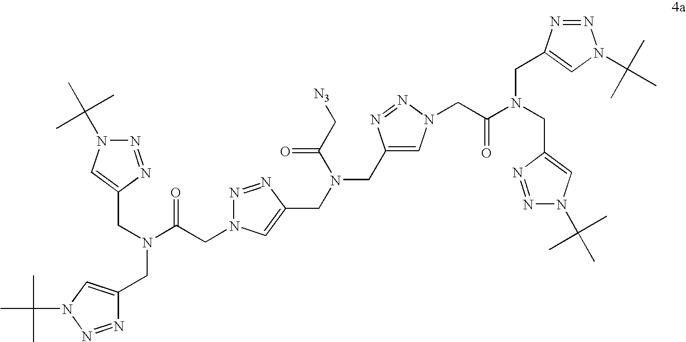

tBu-F-[G-2]-N₃, 4a: ¹H NMR (500 MHz, [D6]Acetone): δ=8.16 (s, 1H), 8.14 (s, 1H), 7.89 (s, 1H), 7.86 (s, 1H), 7.80 (s, 1H), 7.78 (s, 1H), 5.77 (d, 4H), 4.80 (s, 2H), 4.77 (s, 2H), 4.74 (s, 2H), 4.67 (s, 2H), 4.60 (d, 4H), 4.41 (s, 2H), 1.68 (s, 9H), 1.67 (s, 9H), 1.63 (s, 18H). ¹³C NMR (125 MHz, [D6]Acetone): δ=168.7, 166.8, 166.6, 144.2, 143.8, 143.7, 143.3, 126.2, 126.1, 121.4, 60.6, 60.1, 59.9, 52.1, 51.2, 42.6, 41.8, 41.5, 30.2 ppm. MALDI-FTMS: expect MH⁺ 925.5353, found 925.5368.

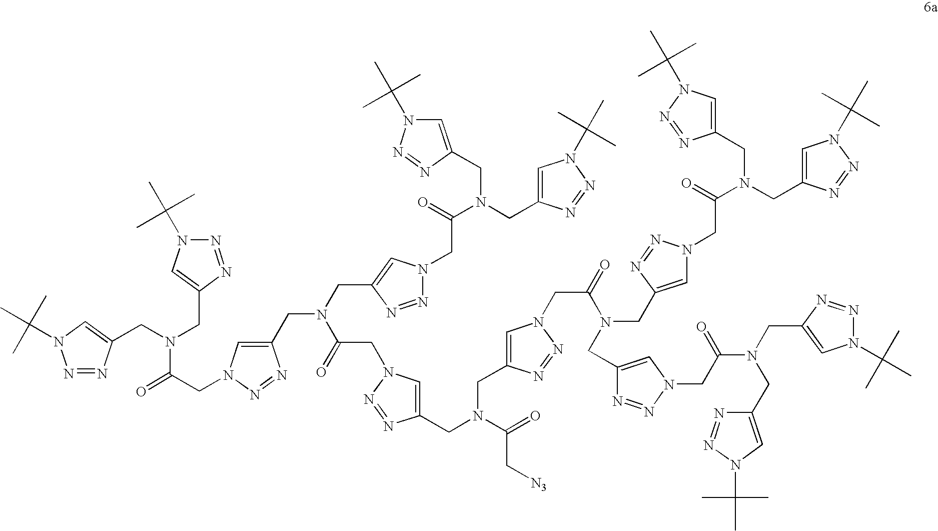

tBu-F-[G-3]-N₃, 6a: ¹H NMR (600 MHz, [D6]DMSO): δ=8.29 (d, 4H), 8.17 (d, 2H), 8.14 (s, 1H), 8.03 (d, 4H), 8.00 (s, 1H), 7.96 (d, 2H), 5.76 (m, 12H), 4.77 (s, 4H), 4.72 (d, 8H), 4.56 (t, 16H), 4.40 (s, 2H), 1.61 (s, 36H), 1.57 (s, 36H). ¹³C NMR (150 MHz, [D6]Acetone): δ=168.2, 166.6, 166.4, 166.3, 166.2, 143.5, 143.4, 143.3, 143.1, 142.7, 125.8, 120.9, 59.5, 59.3, 51.6, 51.5, 50.6, 42.2, 41.9, 41.4, 40.8, 29.5 ppm, 29.1. MALDI-TOF: 2026 (MH⁺), 2048 (MNa⁺), PDI: 1.005.

Synthesis of dendrimer 7a: 160.2 mg (0.08 mmol) of tBu-F-[G-3]-N₃ 6a is mixed with 6.4 mg (0.026 mmol) of 2,4,6-tris-prop-2-ynyloxy-[1,3,5]triazine 23. The mixture is diluted with 0.8 ml of 1:1 tBuOH:H₂O solution. Sodium ascorbate (3.1 mg, 0.016 mmol, 0.20 eq) is added as a solid, followed by the addition of CuSO₄ (2 mg, 0.008 mmol, 0.10 eq). The reaction is stirred at room temperature and completed overnight as indicated by LC-MS. The reaction mixture is diluted with 5 ml H₂O and 1 ml concentrated NH₄OH/citrate buffer, stirred for 2 minutes and extracted 3 times with 30 ml portions of CHCl₃. The organic layer is washed with brine, dried over NaSO₄, and evaporated to yield a while solid, which is then purified by prep-HPLC (pump flow gradient settings—solvent CH₃CN/H₂O; flowing rate: 6.5 ml/min, 0 min, 29% CH₃CN; 2 min, 58% CH₃CN, 30 min 80% CH₃CN) to give pure dendrimer 7a 150 mg, 90% yield. ¹H NMR (600 MHz, [D6]DMSO) δ=8.26 (m, 16H), 8.20 (s, 3H), 8.15 (d, 6H), 8.00 (m, 15H), 7.95 (d, 5H), 5.76 (m, 44H), 5.51 (s, 6H), 4.74 (m, 44H), 4.55 (m, 44H), 1.59 (dd, 108H), 1.54 (dd, 108H). ¹³C NMR (150 MHz, [D6]Acetone) δ 167.3, 143.9, 143.6, 143.4, 143.1, 126.8, 122.2, 121.9, 119.3, 113.6, 60.7, 60.4, 52.4. 42.7, 41.4, 30.1. MALDI-TOF: calcd for (C₂₇₆H₃₉₃N₁₅₉O₂₄+Na)⁺: 6345, found: 6345±0.1%, PDI: 1.027.

1. A process for producing a product dendron having a single azide group, the process comprising the following steps:
Step A: reacting “n” organic azide molecules with an ABₙ molecule, the ABₙ molecule having “n” terminal acetylene functionalities and one halomethyl group, “n” being two or greater, the reaction occurring in the presence of sufficient copper catalyst to insure complete reaction for producing a product molecule having “n” triazoles and one halomethyl group; and then
Step B: reacting the product molecule of said Step A with sufficient sodium azide in an organic/aqueous solvent mixture at a temperature high enough to give complete or nearly complete displacement of the chloride from the halomethyl group for producing the product dendron having a single azide group.

2. A process according to claim 1 wherein the product dendron is a first generation dendron.

3. A process according to claim 2 wherein the organic azide is selected from the group represented by the following structures:

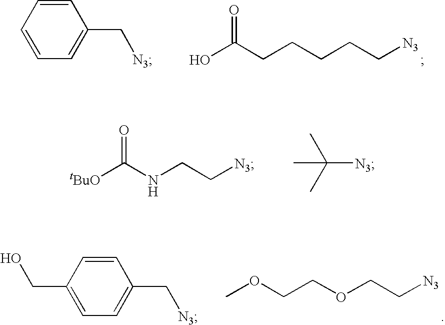

4. A process according to claim 1 wherein “n” is two.

5. A process according to claim 4 wherein the ABₙ molecule of Step A is selected from the group represented by the following structures:

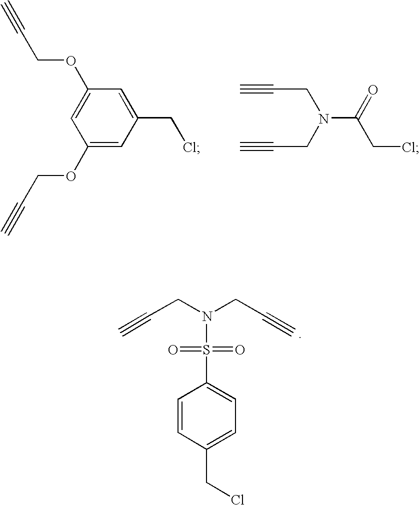

6. A process according to claim 1 wherein the product dendron is a second generation dendron and each of the “n” organic azide molecules is a first generation dendron.

7. A process according to claim 6 wherein each of the “n” organic azide molecules is the product dendron of claim 2.

8. A process according to claim 1 wherein the product dendron is a third generation dendron and each of the “n” organic azide molecules is a second generation dendron.

9. A process according to claim 8 wherein each of the “n” organic azide molecules is the product dendron of claim 6.

10. A process according to claim 1 wherein the product dendron is a fourth generation dendron and each of the “n” organic azide molecules is a third generation dendron.

11. A process according to claim 10 wherein each of the “n” organic azide molecules is the product dendron of claim 8.

12. A process for producing a triazole containing dendrimer, the process comprising the following step:
Step A: reacting two or more dendrons, each dendron possessing a single azide functionality, with a polyacetylene core compound, the polyacetylene core compound containing two or more terminal acetylene groups, in a suitable solvent and in the presence of catalytic quantity of copper(I) species for catalyzing a triazole formation reaction for forming the dendrimer.

13. A process according to claim 12 comprising the following further step:
Step B: washing the product of said Step A with sufficient aqueous ammonium hydroxide/citrate solution to remove copper species that may be bound to triazole moieties of the dendrimer.

14. A process according to claim 12 wherein the polyacetylene core compound is selected from the group represented by the following structures:

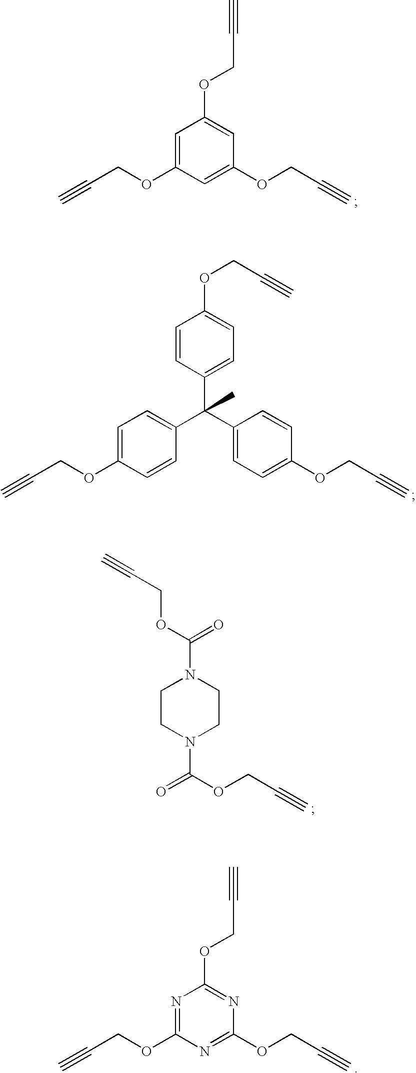

15. A process for producing a first generation dendrimer according to claim 12 wherein the dendron used in said Step A is a first generation dendron.

16. A process for producing a second generation dendrimer according to claim 12 wherein the dendron used in said Step A is a second generation dendron.

17. A process for producing a third generation dendrimer according to claim 12 wherein the dendron used in said Step A is a third generation dendron.

18. A process for producing a fourth generation dendrimer according to claim 12 wherein the dendron used in said Step A is a fourth generation dendron.

19-26. (canceled)

27. A trifunctional reagent represented by the following formula: 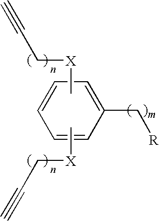
wherein:
X is a diradical selected from the group consisting of —O— and —S—;
R is a radical selected from the group consisting of —Cl and —Br;
n is 1-10; and
m is 1-10.

28. A trifunctional reagent according to claim 27 represented by the following formula:

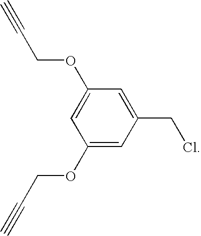

29. A trifunctional reagent represented by the following formula: 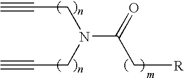
wherein:
R is a radical selected from the group consisting of —Cl and —Br;
n is 1-10; and
m is 1-10.

30. A trifunctional reagent according to claim represented by the following formula:

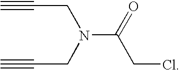

31. A trifunctional reagent represented by the following formula: 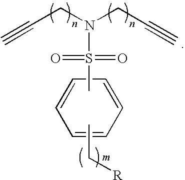
wherein:
R is a radical selected from the group consisting of —Cl and —Br;
n is 1-10; and
m is 1-10.

32. A trifunctional reagent according to claim represented by the following formula:

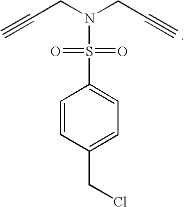

33. A core molecule represented by the following formula: 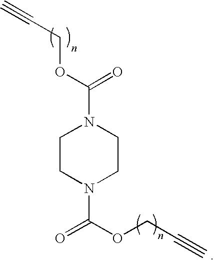
wherein:
n is 1-10.
